(19) United States
(12) Patent Application Publication
(10) Pub. No.: US 2004/0171964 A1
(43) Pub. Date: Sep. 2, 2004

(54) Device for family planning and preventing conception

(76) Inventor: Kilian Heitz, Bonstetten (CH)

Correspondence Address: FULBRIGHT & JAWORSKI, LLP, 666 FIFTH AVE, NEW YORK, NY, 10103-3198, US

(21) Appl. No.: 10/714,072

(22) Filed: Nov. 14, 2003

(30) Foreign Application Priority Data
Nov. 14, 2002 (DE) 102 53 335.0

(51) Int. Cl. A61B 10/00

(52) U.S. Cl. 600/551

(57) ABSTRACT

The present invention involves a device for family planning and/or countraception, whereby the device has a means for determining basal temperature, a means for input of the first day of the cycle, a means for input of the property of the saliva, a device for processing the data made available by the means, a memory device for storing at least some of the data made available and a display device, whereby the device for processing the data is designed such that a conclusion regarding the woman's fertility on any given day is determined as a function of at least some of the data made available by each means and this conclusion can be displayed via the display device.

Various contraceptive methods have been developed and used in the past. The most widely used methods are mechanical, such as condoms or IUDs, hormonal (pill) and those of a chemical nature, such as suppositories, creams and foam. Devices have recently been developed for both contraception and family planning which are capable of evaluating the probability of conception on a certain day. A woman's fertility varies over her cycle, so that one egg matures approximately every 28 days to the extent that it is released by the ovary and can be fertilized. The release of the egg is an event known as ovulation. The ovum is fertile for only about 12 to at most 24 hours after ovulation. However, sperm cells are viable in a woman's body for an average of 72 hours or in exceptional cases up to five days. The period of time of maximum fertility therefore begins approximately 72 hours before ovulation and ends approximately 12 hours afterward. All known methods of determining the fertility phase therefore attempt to predict the time of ovulation because the probability of conception is greatest around the time of ovulation. Within a cycle, many parameters vary, such as the woman's body temperature or basal temperature, LH hormone level and the properties and amount of the cervical mucus as a function of the phase of the cycle. On the basis of these parameters, it is theoretically possible to develop methods which will make it possible to determine the point in time of ovulation and thus the fertile days of a woman.

It is known that the female cycle is subject to a complex hormonal control. The average duration of the cycle is 28 days but it varies between 24 and 32 days or may even be irregular. A known method of determining a woman's fertility period is the so-called calendar method, which assumes that ovulation occurs regularly at the middle of the cycle. It is assumed here that the duration of the cycle is 28 days and that ovulation occurs 14 days after the first day of the last cycle. However, since each cycle is subject to certain fluctuations, the calendar method is inaccurate and therefore is not suitable for reliable contraception or family planning. WO 01/36212 describes a device which is based exclusively on the calendar method and also determines the point of ovulation and thus of a woman's fertility.

In addition, measurement of the basal temperature is a widely used method of determining ovulation. After each ovulation, the sex hormone gestagen is formed; this has the property of acting on the heat regulation center so that the body temperature rises about 0.4 to 0.6° C. As a result, the average temperature in the first half of the cycle (before ovulation) is lower than the average temperature in the second half of the cycle (after ovulation). Since gestagen is formed only when ovulation has already occurred, the rise in temperature may be considered a reliable sign of ovulation. However, if there are factors such as a cold or a migraine, these may also result in an elevated body temperature and then it is difficult or even impossible to determine the time of ovulation. This shows that use of the basal temperature method as a low degree of accuracy and does not allow any prediction of ovulation.

European Patent 0 195 207 describes a device for regulating contraception, having a temperature measurement device for measuring body temperature, whereby the temperature measurement device is accommodated in a housing which can be inserted into the body, and this housing also contains a data memory which stores the temperature in fixed intervals with the control of a timer, and a transmitting and receiving device is provided to send data out of the data memory in response to an external command signal. WO 87/02876 describes a device for determining the basal temperature curve and for determining the days of conception, with a device for measuring the body core temperature, an analyzer device in which the body core temperature values measured each day can be stored in correlation with the measurement date and analyzed, and with a display and operating unit as well as optionally a clock and alarm function, whereby in order to determine the time of contraception, the analyzer unit first compares a number of successive measured temperature values, the number of which is preselectable, with a starting temperature value determined by it from the temperature value series measured at the beginning of the cycle; that more than half of the temperature values measured at the beginning of the cycle are at least 0.1° C. lower than the starting temperature value; and when more than half of the temperature values of this series measured most recently are greater than the starting value, the analyzer unit checks, by using a second temperature value which is higher by 0.1° C. than the starting value, to ascertain in which series of temperature values shifted from the start of the cycle to the end of the cycle and/or to the current measurement date, more than half of the measured temperature values are equal to or greater than the second temperature value for the first time, and it defines the date of the measurement of the first temperature value of this series, which is at least at the level of the second temperature value, as the midpoint of a new series in which the minimum representing the date of ovulation is calculated, whereby when there are multiple minimums, the one closest to the end of the cycle is considered to be the date of ovulation.

Another method of determining ovulation is the cervical mucus method developed by Professor Billing. At the start of the cycle, after menstruation, a small amount of cervical mucus is discharge. Around the time of ovulation, the cervical glands produce mucus which is initially thick and becomes progressively clearer and more liquid in the following days. It is assumed that the fertile days begin with the appearance of the mucus and last for several days. Then the mucus becomes viscous again and the fertility interval is past. However, the natural properties of cervical mucus may be altered through the use of chemical contraceptives or other circumstances. In addition to the cervical mucus, saliva also changes during these cycles so this change may also be used as a basis for determining whether or not ovulation has occurred. WO 01/06932 describes a device by which the saliva or cervical mucus can be tested to determine the fertile time.

Ovulation can also be detected by determining the concentration of the luteinizing hormone (LH) in urine, and there are numerous devices on the market using this method. Around the time of ovulation there is an increase in LH, which is a good indicator for the time of ovulation. The device for determining the LH concentration includes test strips with monoclonal antibodies that are specific for LH. Subsequent ELISA or EMIT method then makes it possible to determine the concentration of LH on the basis of a color change. By daily use of this method, it is possible to determine whether the LH concentration increases from one day to the next. European Patent 0 383 619 describes a kit with test strips for determining the urinary concentration of LH. The method is much more reliable than the methods mentioned first, but it is complicated to use and the test strips are expensive.

All the methods mentioned above can be used to determine whether ovulation has occurred. However, none of these methods alone can predict exactly the time of ovulation. Therefore, the devices available on the market include one or more devices which make it possible to perform several methods to make a prediction as to when the time of the next ovulation will be. The reliability of a contraception method is given by the Pearl Index, and most devices available on the market yield a value between 0.5 and 7 on the Pearl Index (the lower the value, the more reliable the device). The accuracy of the available devices depends on the measurement method used. The problem is that the methods either have a low level of reliability or they are comparatively complicated to use when greater accuracy is desired. The object of the present invention is to provide a device which will combine simple handling with great accuracy in prediction and determination of ovulation and in doing so will not require any expensive components such as test strips with monoclonal antibodies. The object of the present invention is to provide such a device.

The inventive device combines the following methods for determining the presumed point in time of ovulation: the calendar method, the basal temperature method and the saliva method.

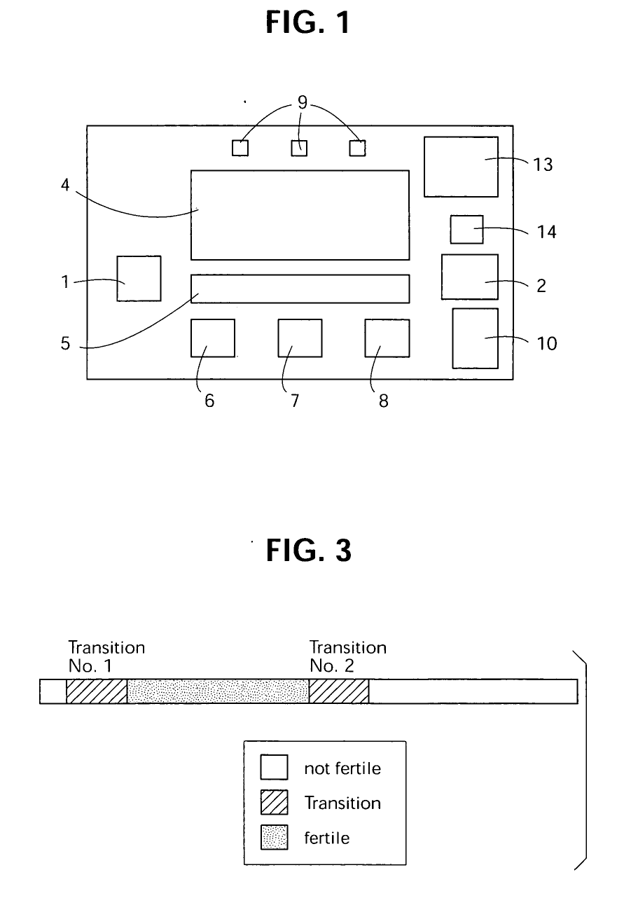

This object is achieved by the method according to claim 1. The embodiment according to claim 1 is a device for family planning and/or contraception whereby the device has a means (1) for determining the basal temperature, a means (7) for input of the first day of the cycle, a means (8) for input of the properties of the saliva, a device for processing the data (13) made available by the means (1, 7, 8), a memory device (14) for storing at least some of the data (14) made available and a display device (4), whereby the device for processing the data is designed such that a conclusion regarding the woman's fertility on any given day is determined as a function of at least some of the data made available by each means and this conclusion can be displayed via the display device (see FIG. 1).

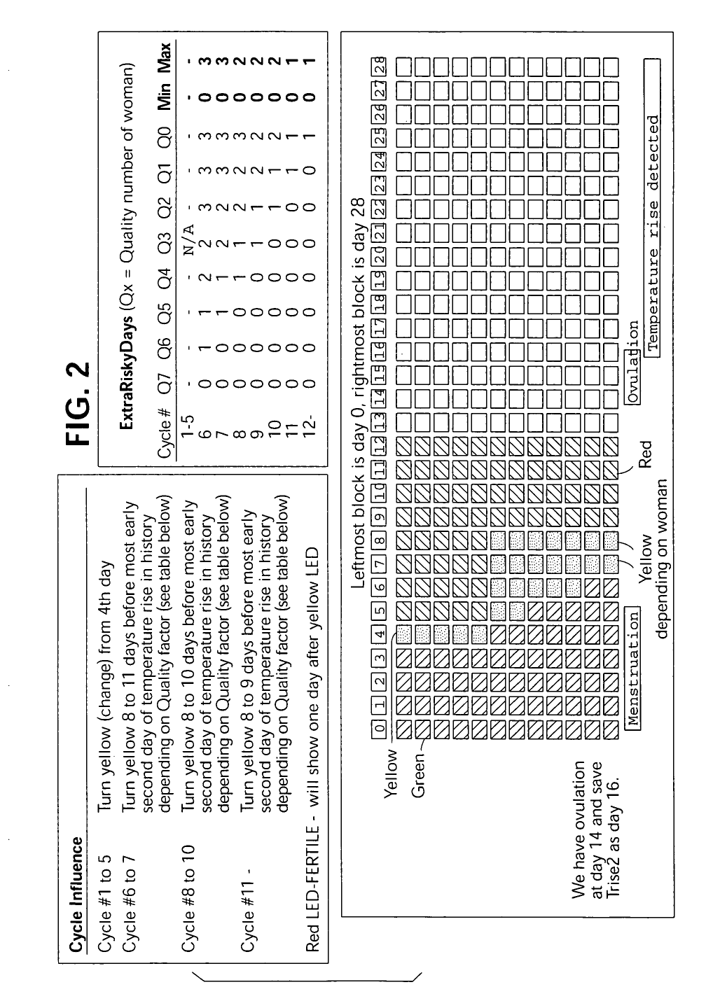

For example, only data on the basal temperature during the first cycle or the first and second cycle may be taken into account, to achieve the greatest possible certainty with regard to a conclusion about a woman's fertility on any given day in the initial stage of use of the inventive device. In a preferred embodiment, the display device displays fixed values at the beginning of use of the device, for example, four possible infertile days, followed by a day on which a conclusion regarding a woman's possible fertility or infertility is impossible, followed by a number of possible fertile days (see FIG. 2).

In another preferred embodiment, fixed values are used up to and including the fifth cycle in the period of time prior to ovulation. Only as of the sixth cycle is the regularity of the start of the cycle (input of the first day of the woman's period) taken into account in a conclusion regarding her fertility on any given date. For example, this is done by calculating back at least six days from the expected date of ovulation, depending on the regularity of the start of the cycle.

In another preferred embodiment, in addition to the fixed values in the period of time prior to ovulation, fixed values are also used in the period of time after ovulation has occurred; this may be two days, for example (ovulation plus one day). For example, this could result in the conclusion regarding the woman's fertility, yielding a possible fertile day on at least eight successive days.

As of the second cycle, data regarding the properties of the saliva may also be taken into account. However, input of data on the properties of the saliva may also be used only for habituation of the user without it having any influence on the analysis in the current month. In another embodiment, the saliva measurement has an influence on the conclusion regarding the woman's fertility on any day as of the third cycle, so that, for example, the number of possible fertile days after ovulation might be reduced.

The device according to claim 1 is also characterized in that the device for processing the data is designed so that the data is weighted differently in determining the conclusion regarding the woman's fertility, in which case the weighting of the data can be varied as a function of the stored data formerly made available.

According to a special embodiment, the inventive device is characterized in that the conclusion regarding the woman's fertility depends on the data made available by the means (1) for determining the basal temperature to a greater extent than on the data made available by the means (7) for input of the first day of the woman's cycle, and the data made available by the means (7) for input of the first day of the woman's cycle in turn has a greater influence on the conclusion regarding her fertility than does the data made available by the means (8) for input of the properties of the saliva.

According to another special embodiment, the inventive device is characterized in that the weighting of the data made available by the means (1) for determining the basal temperature preferably amounts to 50-90%, especially preferably 60-80%; the weighting of the data made available by the means (7) for input of the first day of the woman's cycle preferably amounts to 5-35%, especially preferably 10-30%, and weighting of the data made available by the means (8) for input of the properties of the saliva preferably amounts to 2-20%, especially preferably 5-15%.

The calendar method is weighted less according to this particular embodiment than the temperature method, because the calendar method is less reliable than the temperature method because of the natural fluctuations in cycle. The weighting of the saliva test method is lower than the weighting of the calendar method because even if the calendar method is not very reliable, the calendar still is an objective method while the saliva test method is very subjective.

Because of the combination of three methods, the inventive device is capable of recognizing fluctuations in temperature that are not due to the cycle but instead are caused by other factors such as a cold or a migraine and then to discard this data due to temperature fluctuations when determining the possible fertile and/or infertile days.

One particular feature of the inventive device is that it is not necessary for all measurements to be performed every day. In one particular embodiment, the inventive device is characterized in that it includes a time measuring device and an interactive device for instructing a user to input data after a predetermined period of time has elapsed. In another embodiment, the inventive device demands by way of an interactive device input of the quantity and properties of the saliva preferably 5-10 times per cycle, especially preferably 6-8 times per cycle.

In another embodiment, the device is automatically capable of recognizing a cycle and in particular the duration and/or the course of the cycle. Therefore, the maximum accuracy is preferably reached before the sixth or seventh cycle, especially preferably before the fifth cycle.

The advantages of the inventive device include its simple handling, its clear, unmistakable displays and instructions and the fact that it is controlled by a modern microprocessor.

In addition, the present invention includes the applications and a method for using the inventive device.

This device is used for determining the ovulation phase by means of the calendar method, the basal temperature method and the saliva method, and these values are compared. This device is used for family planning, i.e., for preventing contraception or for fulfilling the desire for a child. The device is preferably used several times per week, especially preferably once a day.

This device is equipped for processing data, such that the conclusion regarding a woman's fertility differentiates between possible fertile and infertile days and that the possible fertile and infertile days are displayed visually in different ways, with the display device preferably showing the possible fertile days in red and the possible infertile days in green.

This device is also characterized in that so-called transition days are also provided in the conclusion regarding the woman's fertility; these are days when no distinction is made between possible fertile days and possible infertile days, and the transition days are displayed differently visually than the possible fertile days and infertile days. In a preferred embodiment, the display device displays the transition days in yellow.

In another embodiment, the device is characterized by a means for recognizing an increase in temperature from the data made available by the means (1) for determining the basal temperature, whereby in determining the conclusion regarding the woman's fertility, a distinction is made between a first phase of the cycle at the beginning of the cycle and a second phase of the cycle after the rise in temperature is detected.

The terms “first phase of the cycle” and “second phase of the cycle” are understood to mean that the first phase of the cycle is the first period of time from the start of the cycle. This first phase of the cycle includes possible infertile days and the transition to possible fertile days. The second phase of the cycle includes the transition from possible fertile days to possible infertile days after the rise in temperature has occurred (see FIG. 3).

In another embodiment of the device, up to a first point in time, for example, the end of the second cycle registered by the device, the number of possible fertile days is preselected in the conclusion regarding the woman's fertility in the second phase of the cycle. After the first point in time, the number of possible fertile days is determined as a function of an analysis of stored data in the conclusion regarding the woman's fertility in the second phase of the cycle.

In a preferred embodiment, the device is characterized in that an indicator for the regularity of the duration of the cycle and/or the course of the cycle is derived from the stored data, and the number of possible infertile days is determined as a function of this indicator and/or the number of cycles registered by the device is determined in the conclusion regarding the woman's fertility in the second phase of the cycle.

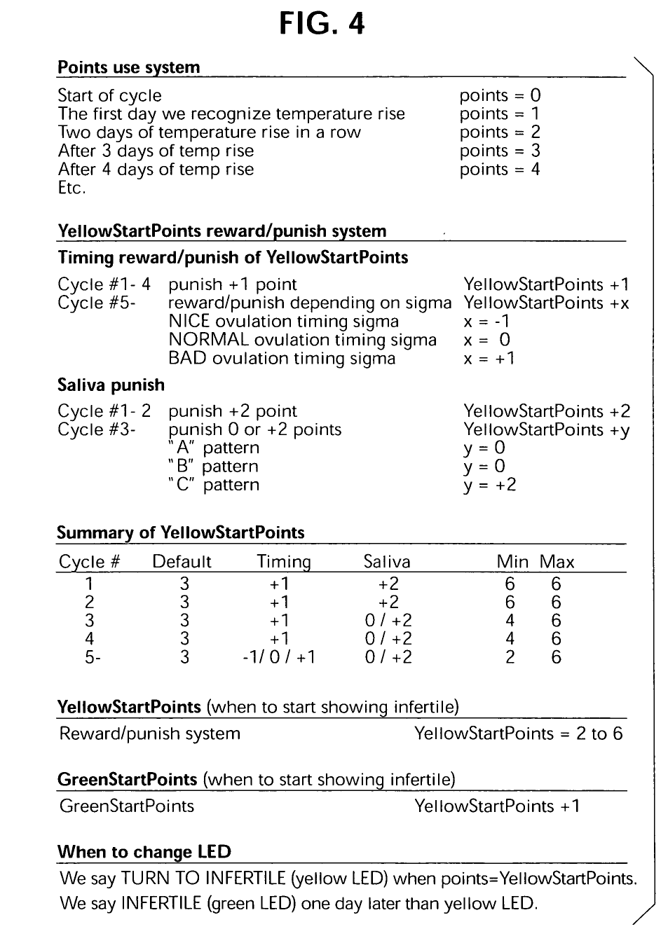

The indicator according to the present invention is, for example, the standard deviation (sigma, a) of the day for the date of ovulation (rise in temperature). The date on which the ovulation of the next cycle occurred is related to the average of the days on which the ovulation of the preceding cycles occurred (see FIG. 4). “Good” denotes a sigma of 0.8 of the average. “Bad” denotes a sigma of more than 2.0, and “normal” denotes a sigma between 0.8 and 2.0. Sigma is preferably calculated after the fourth cycle. There is no limit to the number of stored days on which ovulation has occurred. It is preferable to store the last 6 to 24 cycles, preferably the last 15 cycles.

In another embodiment, the number of possible fertile days is determined and/or influenced as a function of the data made available by the means (8) for input of the properties of the saliva in the conclusion regarding fertility in the second phase of the cycle.

In another embodiment, the inventive device is characterized in that up to a second point in time, e.g., the end of the fifth cycle registered by the device, the number of possible infertile days is preselected in the conclusion regarding the woman's fertility in the first phase of the cycle, and preferably after the second point in time, the number of possible infertile days is determined as a function of an analysis of stored data in the conclusion regarding the woman's fertility in the first phase of the cycle.

In a particularly preferred embodiment, the inventive device is characterized in that a quality factor is derived from the stored data, and after the second point in time, in the conclusion regarding the woman's fertility in the first phase of the cycle, the number of possible infertile days is determined as a function of the quality factor and/or the number of periods registered by the device is determined as a function of some data from the means (7) for input of the first day of the cycle.

The quality factor is calculated by the computer program of the inventive device (ixP2σ) and is an indicator of the constancy of the time of ovulation in all measured cycles in the range of 0 to 7. For example, if ovulation always occurs on day 14, then there is a very high quality factor, i.e., a quality factor=7.

0≦ixP2σ<0.5 yields a quality factor of 7; 0.5≦ixP2σ<1.0 yields a quality factor of 6, etc. A low quality factor is an indicator of a great variation in the time of ovulation. 3.0≦ixP2σ<3.5 yields a quality factor of 1, and 3.5≦ixP2σ yields a quality factor of 0.

This method is used for determining the ovulation phase and/or for family planning. By means of the calendar method, the basal temperature method and the saliva method, parameters of the female body are measured, and then these values are compared. The measured parameters preferably include the body temperature and the consistency of the saliva and/or the cervical mucus, whereby the body temperature is preferably measured orally and the consistency of the saliva is preferably measured outside of the body. This data is then stored, preferably using a microprocessor.

This invention will now be explained in greater detail on the basis of the figures, which show:

FIG. 1: a block diagram of the inventive device.

FIG. 2: transition no. 1 during its use over several cycles.

FIG. 3: the course of the phases in the cycle in the display device.

FIG. 4: transition no. 2.

FIG. 5: the back side of the inventive device.

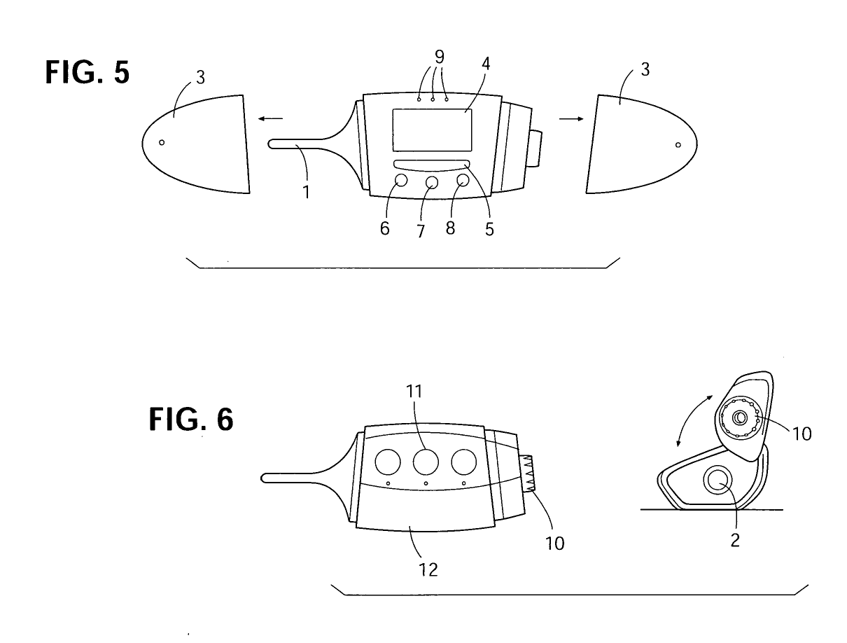

FIG. 6: a view of the inventive device.

FIG. 1 shows a block diagram of the inventive device.

FIG. 2 shows transition no. 1 during its use over several cycles. Transition no. 1 is the first part of the cycle at the beginning of the cycle (bleeding has just occurred). The user is infertile for three days, so the display device displays “possibly infertile” for four days, including day zero (before the first temperature measurement). As of the fourth day, there is a transition from the display “possibly infertile” over a period of several days to the display “possibly fertile” as a function of when there must be a transition to “possibly fertile” in order to make the most reliable possible conclusion regarding the woman's fertility, e.g., when the device is used for contraception. The data is processed with regard to the greatest possible certainty. It is assumed that sperm has a survival time of five days and that the egg cell remains fertilizable for up to two days after ovulation. Data on the start of the cycle of previous cycles is used statistically to determine when there must be a change to “possibly fertile.”

FIG. 3 shows, for example, the course of the phases of the cycle in the display device of the inventive device.

Transition no. 1 is the first part of the cycle at the beginning of the cycle. For example, three possible infertile days are displayed. Then there is a change from possible infertile days to possible fertile days, for example, over a period of ten days. Transition no. 2 is the part of the cycle during which there is a change from possible fertile days to possible infertile days after ovulation. For example, the duration of the possible infertile days displayed may be 12 days.

FIG. 4 shows as an example how an indicator of the regularity of the duration of the cycle is derived for determining transition no. 2.

FIG. 5 shows a view of a preferred embodiment of the inventive process-controlled minicomputer for determining ovulation and/or for family planning. Preferably three tested methods of natural family planning (NFP)—the calendar method, the basal temperature method and the saliva method—are combined appropriately and analyzed using algorithmic software. The result is displayed on the display (4) and can be called up at any time by actuation of the function key (5). By means of the key for cycle input (7) the first day of the last menstrual bleeding is entered so that the device has a first calculation basis according to the calendar method to assign the user to a cycle group. It is even possible to confirm this entry 3 to 5 days after the start of a menstrual cycle. At the latest after the 5^{th} cycle, but preferably after the 3^{rd} cycle, the device will have reached its maximum efficiency, so the device becomes more accurate the longer it is used and a Pearl Index of 0.5-2 is reached. These measurements are preferably performed in the morning before getting up, or at any rate should be performed regularly at the same time of day, so the device includes a built-in alarm which is set for the desired time by operation of the function key (5). To measure the basal temperature, the key for the temperature measurement (6) is depressed and the protective cap (3) over the temperature sensor (1) is removed and the temperature sensor (1) is guided beneath half of the tongue for a few seconds until the measurement is concluded. The end of the measurement is indicated by an optical or an acoustic signal, preferably by an actuation sound. The measured value can be seen on the display (4) and can be stored in the computer by depressing the function key (5). Then the status light (9) lights up, providing information regarding the phase of the cycle. The status light (9) preferably consists of three lights, where green indicates that no conception is possible, yellow indicates a transitional phase where conception would be uncertain and red indicates the ovulation phase as the most reliable time for conception.

The device instructs a second measurement method to be performed 5-10 times per cycle, preferably 6-8 times per cycle, preferably for the saliva test method. Therefore, the display (4) shows a message such as “please perform saliva test,” which is accompanied by a signal, e.g., a warning tone. To measure the saliva, preferably morning saliva, the key (8) for the saliva test is depressed and the protective cap (3) above the saliva carrier (2) is removed. Then this carrier (2) is extracted from the device, the surface of the carrier is cleaned briefly with a cloth and a small amount of saliva from the mouth, preferably morning saliva, is applied to the carrier (2). The carrier (2) is returned to the device, preferably after drying the saliva on the carrier (2). The test saliva is backlit and is then compared with reference images, preferably mounted on the back side of the device, as shown in FIG. 2, to determine the phase of the cycle on the basis of these images. The measured data is stored in the device by operating the function key (5).

FIG. 6 shows the preferred embodiment of the backside of the measurement device, which preferably has 3-5 reference images (11) for the crystal structure of the saliva. The user's saliva, which is on the saliva carrier (2), is observed through an adjustable eyepiece (10) and compared with the images (11). The results of this measurement are then stored in the device by depressing the function key (5), which is on the other side of the device (FIG. 5).

In addition, this side of the device includes a connection and/or a compartment (12) for the power supply to the device. The power supply during operation is supplied by means of a line plug, a power pack or a battery. The charge status of the device is indicated by an LED.

### LIST OF REFERENCE NOTATION

FIG. 1. Block Diagram Showing the Inventive Device

(1) means for determining the basal temperature

(2) saliva carrier

(4) display device

(5) function key

(6) means for input of the temperature

(7) means for input of the first day of the cycle

(8) means for input of the property of the saliva

(9) status lights

(10) eyepiece

(13) device for processing the data made available by the means (1, 7, 8)

(14) memory device

FIG. 5. Front Side of the Measurement Device

(1) means for determining the basal temperature

(3) safety caps

(4) display device

(5) function key

(6) means for input of the temperature

(7) means for input of the first day of the cycle

(8) means for input of the property of the saliva

(9) status lights

FIG. 6. Back Side of the Measurement Device

(2) saliva carrier

(10) pivotable, adjustable eyepiece

(11) comparative images for the saliva

(12) battery cover

1. A device for family planning and/or contraception, whereby the device has
a means (1) for determining basal temperature,
a means (7) for input of the first day of the cycle,
a means (8) for input of the property of the saliva,
a device for processing the data (13) made available by the means (1, 7, 8),
a memory device (14) for storing at least some of the data made available and
a display device (4),
whereby the device for processing the data is designed such that a conclusion regarding the woman's fertility on any given day is determined as a function of at least some of the data made available by each means and this conclusion can be displayed via the display device.

2. The device according to claim 1, characterized in that the device for processing the data is designed so that the data is weighted differently in determining the conclusion regarding the woman's fertility.

3. The device according to claim 2, characterized in that the weighting of the data can be varied as a function of the stored data formerly made available.

4. The device according to one of the claims 2 to 3, characterized in that the conclusion regarding the woman's fertility depends on the data made available by the means (1) for determining the basal temperature to a greater extent than on the data made available by the means (7) for input of the first day of the woman's cycle, and the data made available by the means (7) for input of the first day of the woman's cycle in turn has a greater influence on the conclusion regarding her fertility than does the data made available by the means (8) for input of the properties of the saliva.

5. The device according to one of the claims 2 to 4, characterized in that the weighting made available by the means (1) for determining the basal temperature preferably amounts to 50-90%, especially preferably 60-80%; the weighting of the data made available by the means (7) for input of the first day of the woman's cycle preferably amounts to 5-35%, especially preferably 10-30%, and weighting of the data made available by the means (8) for input of the properties of the saliva preferably amounts to 2-20%, especially preferably 5-15%.

6. The device according to one of the previous claims, characterized in that the device is equipped for processing data, such that the conclusion regarding a woman's fertility differentiates between possible fertile and infertile days and that the possible fertile and infertile days are displayed visually in different ways.

7. The device according to claim 6, characterized in that the display device shows the possible fertile days in red and the possible infertile days in green.

8. The device according to one of the claims 6 to 7, characterized in that transition days are also provided in the conclusion regarding the woman's fertility; these are days when no distinction is made between possible fertile days and possible infertile days, and the transition days are displayed differently visually than the possible fertile days and infertile days.

9. The device according to claim 8, characterized in that the display device displays the transition days in yellow.

10. The device according to one of the claims 6 to 9, characterized by a means for recognizing an increase in temperature from the data made available by the means (1) for determining the basal temperature, whereby in determining the conclusion regarding the woman's fertility, a distinction is made between a first phase of the cycle at the beginning of the cycle and a second phase of the cycle after the rise in temperature is detected.

11. The device according to claim 10, characterized in that up to a first point in time, for example, the end of the second cycle registered by the device, the number of possible fertile days is preselected in the conclusion regarding the woman's fertility in the second phase of the cycle.

12. The device according to claim 11, characterized in that after the first point in time, the number of possible fertile days is determined as a function of an analysis of stored data in the conclusion regarding the woman's fertility in the second phase of the cycle.

13. The device according to claim 12, characterized in that an indicator for the regularity of the duration of the cycle and/or the course of the cycle is derived from the stored data, and the number of possible infertile days is determined as a function of this indicator and/or the number of cycles registered by the device is determined in the conclusion regarding the woman's fertility in the second phase of the cycle.

14. The device according to one of the claims 10 to 13, characterized in that the number of possible fertile days is determined and/or influenced as a function of the data made available by the means (8) for input of the properties of the saliva in the conclusion regarding fertility in the second phase of the cycle.

15. The device according to one of the claims 10 to 14, characterized in that up to a second point in time, e.g., the end of the fifth cycle registered by the device, the number of possible infertile days is preselected in the conclusion regarding the woman's fertility in the first phase of the cycle.

16. The device according to claim 15, characterized in that after the second point in time, the number of possible infertile days is determined as a function of an analysis of stored data in the conclusion regarding the woman's fertility in the first phase of the cycle.

17. The device according to claim 16, characterized in that a quality factor is derived from the stored data, and after the second point in time, in the conclusion regarding the woman's fertility in the first phase of the cycle, the number of possible infertile days is determined as a function of the quality factor and/or the number of periods registered by the device is determined as a function of some data from the means (7) for input of the first day of the cycle.

18. The device according to one of the previous claims, characterized by a time measuring device and an interactive device for instructing a user to input data after a predetermined period of time has elapsed.

19. The device according to claim 18, characterized in that the interactive device is designed so that an input of the properties of the saliva is instructed preferably 5-10 times per cycle, especially preferably 6-8 times per cycle.

20. The device according to one of the previous claims, designed so that fluctuations in temperature that are not due to the cycle but instead are caused by other factors such as a cold or a migraine are recognized and then to discard this data when determining the possible fertile and/or infertile days.

21. The device according to one of the previous claims, designed so that a cycle and in particular the duration and/or the course of the cycle are recognized.
